(19) United States
(12) Patent Application Publication
(10) Pub. No.: US 2006/0029649 A1
(43) Pub. Date: Feb. 9, 2006

(54) Stabilized antimicrobial agents

(76) Inventors: Ahmed Tafesh, Allentown, PA (US); Maria Alonso-DeBolt, Budd Lake, NJ (US)

Correspondence Address: Robert F. Tavares;c/o Troy Corporation, 8 Vreeland Road, Florham Park, NJ, 07932-0955, US

(21) Appl. No.: 10/913,046

(22) Filed: Aug. 6, 2004

(51) Int. Cl. A61K 31/325; A61K 9/70

(52) U.S. Cl. 424/443; 442/123; 514/479

(57) ABSTRACT

The present invention provides antimicrobial compositions comprising an antimicrobial agent and a thermally stabilizing effective amount of an amide based stabilizing agent. The present invention also provides polymeric-cellulosic fiber formulations comprising a polymeric-cellulosic fiber composition and an antimicrobially effective amount of the antimicrobial composition. The present invention further provides a method of thermally stabilizing an antimicrobial agent and an improved method for making a polymeric-cellulosic fiber formulation with antimicrobial activity.

### FIELD OF THE INVENTION

The present invention provides antimicrobial compositions comprising an antimicrobial agent and a stabilizing effective amount of an amide based stabilizing agent. The present invention also provides polymeric-cellulosic fiber formulations comprising a polymeric-cellulosic fiber composition and an antimicrobially effective amount of the antimicrobial composition. The present invention further provides a method of thermally stabilizing an antimicrobial agent and an improved method for making a polymeric-cellulosic fiber formulation that resists antimicrobial degradation.

### BACKGROUND OF THE INVENTION

When exposed to environmental conditions, exterior and interior materials are subject to attack by microorganisms such as fungi, algae, bacteria, and protozoa. Consequently, there is a need for an effective and economical method to protect these materials from destruction by such microorganisms. Such materials include stucco, concrete, stone, cement, wood, caulking, sealants, coating compositions, leather, plastics, textiles, biodegradable compositions, and polymeric-cellulosic fiber formulations.

The rising cost of wood as well as the cost of maintaining the appearance of such wood products has prompted a need to find good quality wood substitutes which are economical and which require minimal maintenance. As a result, a market has emerged for the use of polymeric-wood composites or polymeric-cellulosic fiber formulations to replace traditional natural wood products in applications such as decks, windows, and fences. Polymeric-cellulosic fiber formulations are described in detail in U.S. Pat. Nos. 6,011,091, 6,066,680, and 6,103,791, which disclosures are hereby incorporated by reference. Wood substitutes are generally less susceptible to attack by microorganisms and are thus more durable than wood but still require some degree of protection from destructive microorganisms.

Over the years, a wide variety of antimicrobial agents have been developed to retard or prevent the growth of destructive microorganisms. Such antimicrobial agents include halogenated compounds, organometallic compounds, quaternary ammonium compounds, phenolic compounds, metallic salts, heterocyclic amines, formaldehyde donors, and organo-sulfur compounds.

One of the most significant and effective antimicrobial agents developed are compounds containing a halopropynyl moiety. These are described in detail in U.S. Pat. Nos. 3,660,499; 3,923,870; 4,259,350; 4,592,773; 4,616,004, and 4,639,460, which disclosures are hereby incorporated by reference. Halopropynyl carbamates are especially known for their fungicidal activity and 3-iodo-2-propynyl butyl carbamate (IPBC) is one of the most widely used fungicidal agents. 3-Iodo-2-propynyl butyl carbamate is described in detail in Great Britain patent no. 2,138,292 and U.S. Pat. Nos. 4,915,909 and 5,082,722, which disclosures are hereby incorporated by reference.

Antimicrobial agents are often formulated, and need to be compatible with, a variety of other ingredients that go into the final product to be protected. They may also be combined with other materials to make a new antimicrobial product which has special properties. It is essential that these antimicrobial agent compositions maintain their antimicrobial activity during any formulation or processing that may be required. For example, such antimicrobial compositions may be exposed to high temperatures during extrusion of a polymeric-cellulosic fiber formulation, and if such high temperatures may ordinarily cause the antimicrobial agent to decompose with concomitant loss of antimicrobial activity, measures must be taken to prevent or minimize any such loss.

### BRIEF DESCRIPTION OF THE INVENTION

This invention provides an antimicrobial composition comprising an antimicrobial agent of the halopropynyl type and a thermally stabilizing effective amount of an amide based stabilizing agent. The thermal stability of the halopropynyl antimicrobial agent is improved by the presence of the amide based stabilizing agent.

This invention also provides a polymeric-cellulosic fiber formulation comprising a polymeric-cellulosic fiber composition and an antimicrobially effective amount of the antimicrobial composition. The antimicrobial composition comprises a halopropynyl antimicrobial agent and a thermally stabilizing effective amount of an amide based stabilizing agent to thermally stabilize the antimicrobial agent during processing and thereby impart antimicrobial protection to the polymeric-cellulosic fiber formulation.

This invention further provides a method of thermally stabilizing an antimicrobial agent, which comprises admixing the antimicrobial agent with a thermally stabilizing effective amount of the amide based stabilizing agent.

This invention still further provides an improved method for making a polymeric-cellulosic fiber formulation with antimicrobial resistance, which comprises adding an antimicrobially effective amount of an antimicrobial composition to a polymeric-cellulosic fiber composition. The antimicrobial composition comprises an admixture of a halopropynyl antimicrobial agent and a thermally stabilizing effective amount of an amide based stabilizing agent, to thermally stabilize the antimicrobial agent and thereby impart antimicrobial protection to the polymeric-cellulosic fiber formulation.

### DETAILED DESCRIPTION OF THE INVENTION

Applicants have discovered that antimicrobial agents, particularly 3-iodo-2-propynyl butyl carbamate (IPBC), can be thermally stabilized by admixing the antimicrobial agent with a thermally stabilizing effective amount of an amide based stabilizing agent. While not wishing to be bound by theory, applicants believe that the stabilizing agent and the antimicrobial agent are subject to an attractive interaction which assists in shielding the antimicrobial agent from the normally deleterious effects of elevated temperatures such as those employed during extrusion processes. This shielding retards or prevents decomposition of the antimicrobial agent and results in greater retention of antimicrobial protection in the final product than is the case when the stabilizing agent is not present. The combination of the antimicrobial agent and the thermally stabilizing effective amount of the stabilizing agent work in an unexpected manner to improve the thermal stability of the antimicrobial agent.

In accordance with the present invention, a novel antimicrobial composition is provided which comprises a halopropynyl antimicrobial agent and a thermally stabilizing effective amount of an amide based stabilizing agent.

The halopropynyl compounds that can be used in the present invention may be represented by the formula:
YC≡C—CH₂X
wherein Y is a halogen, preferably iodine; and X is oxygen, nitrogen, sulfur, or carbon, each of which is independently part of an organic functional group. The functional group of which oxygen is a part is preferably an ether, ester, or carbamate group. The functional group of which nitrogen is a part is preferably an amine, amide, urea, nitrile, or carbamate group. The functional group of which sulfur is a part is preferably a thiol, thiane, sulfone, or sulfoxide group. The organic functional group of which carbon is a part is preferably an ester, carbamate, or alkyl group.

The fungicidally active iodopropynyl derivatives useful in the present invention include compounds derived from propynyl or iodopropynyl alcohols such as the esters, ethers, acetals, carbamates and carbonates, and the iodopropynyl derivatives of pyrimidines, thiazolinones, tetrazoles, triazinones, sulfamides, benzothiazoles, ammonium salts, carboxamides, hydroxamates, and ureas. Iodopropynyl derivatives are described in detail in U.S. Pat. Nos. 3,923,870, 4,259,350, 4,592,773, 4,616,004, 4,719,227, and 4,945,109, the disclosures of which are hereby incorporated by reference.

The iodopropynyl carbamates useful in the present invention may be represented by the formula:
[IC≡C—(CH₂)ₘ—O—CO—NH]ₙ—R
wherein R may have one to three linkages corresponding to n and is selected from the group consisting of hydrogen, substituted and unsubstituted alkyl groups having from 1 to 20 carbon atoms; substituted and unsubstituted aryl, alkylaryl, and aralkyl groups having from 6 to 20 carbon atoms; and substituted and unsubstituted cycloalkyl and cycloalkenyl groups having from 3 to 10 carbon atoms. m and n are independently integers from 1 to 3.

The preferred iodopropynyl carbamates useful in the invention are those compounds represented by the following formula, wherein m and n are both 1:
IC≡C—CH₂—O—CO—NH—R
Suitable R substituents include alkyls such as methyl, ethyl, propyl, n-butyl, t-butyl, pentyl, hexyl, heptyl, octyl, nonyl, decyl, dodecyl, and octadecyl; cycloalkyls such as cyclohexyl; aryls, alkaryls, and aralkyls such as phenyl, benzyl, tolyl, and cumyl; halogenated alkyls and aryls such as chlorobutyl and chlorophenyl; and alkoxy aryls such as ethoxyphenyl.

Preferred iodopropynyl carbamates may be selected from the group consisting of 3-iodo-2-propynyl propyl carbamate, 3-iodo-2-propynyl butyl carbamate, 3-iodo-2-propynyl hexyl carbamate, 3-iodo-2-propynyl cyclohexyl carbamate, 3-iodo-2-propynyl phenyl carbamate, and mixtures thereof. More preferably, the iodopropynyl carbamate is 3-iodo-2-propynyl butyl carbamate (IPBC).

The term amide based stabilizing agent, as used in the present invention, is intended to include any compound having amide or amide like groups, i.e. groups that have a —CO—NH— group in the molecule which is sufficiently available to allow the antimicrobial agent to get close enough so that attractive interaction can occur. The term amide based is meant to include carbamates, ureas and the like.

Again, while not intended to be bound by any theory, it appears that the polar nature of the —CO—NH— group can attractively interact with the halopropynyl compound, especially IPBC. This attractive interaction between IPBC and amide or amide like stabilizing agent is believed to be limited only by the ability of the IPBC and amide or amide like group to get close enough to one another to allow the attractive interaction to take place. Once this attractive interactive alignment is allowed to occur, it is believed that the alignment provides a protective shield for the IPBC and slows down the destruction caused by exposures to high temperatures.

When the need for thermal stabilization is connected with extrusion type processes, for example in the manufacture of wood substitutes such as polymer cellulosic fiber plastic composites, it is preferred that the amide based stabilizing agent also be a lubricating agent. Lubricating agents are materials which, in general, are used to improve the internal and external lubricity of materials such as in the manufacture of wood substitutes. [See, for example, U.S. Patent Application Publication No. US 2003/0229160 A1 which discusses the use of lubricants, including amide based lubricants, in the formation of polymer-cellulosic fiber plastic composites, which disclosure is incorporated by reference herein.] For purposes of this invention, the lubricating properties of the amide based lubricating agent may be less important than the stabilizing properties.

The preferred amide based stabilizing agents of this invention that can also serve as lubricating agents are ethylene bisamides represented by the formula:
R—CO—N(H)—CH₂CH₂—N(H)—CO—R
wherein R is independently an alkyl group having from 6 to 16 carbon atoms, preferably from 8 to 14 carbon atoms. Most preferable are those ethylene bisamides that are made from ethylene diamine and readily available fatty acids having C8, C10, C12, and/or C14 carbon atoms. These preferred ethylene bisamides are ethylene bisoctanamide, ethylene bisdecanamide, ethylene bisdodecanamide, ethylene bistetradecanamide, and combinations thereof. The preferred ethylene bisamide lubricating agents are those sold under the trade name Glycolube® WP2200 which is available from Lonza Inc. of Fair Lawn, N.J.

In accordance with the present invention, a thermally stabilizing effective amount of the amide based stabilizing agent is mixed with an antimicrobial agent to prevent or retard loss of the antimicrobial activity of the antimicrobial agent at elevated temperatures. A thermally stabilizing effective amount of a stabilizing agent is an amount effective to prevent or retard the degradation of the antimicrobial agent in the antimicrobial composition at elevated temperatures. As may be expected, an excess of stabilizing agent has been found to more efficiently shield the antimicrobial agent. While antimicrobial compositions containing about 50% antimicrobial agent in the antimicrobial composition were found to exhibit improved thermal stability, more dilute antimicrobial compositions containing about 10% antimicrobial agent in the antimicrobial composition were found to stabilize a higher percentage of the antimicrobial agent. The appropriate amount of the amide based stabilizing agent for a particular purpose can be determined by routine testing of the thermal stability of the antimicrobial agent with varying amounts of added stabilizing agent. Methods for assaying stability of the antimicrobial agents, such as by HPLC, are known and available to one skilled in the art.

The ratio of the antimicrobial agent to the amide based stabilizing agent will depend on the practicalities of use. In a stabilized mixture per se, the objective may be to maximize the amount of antimicrobial agent in the mixture. In the preparation of a polymeric-cellulosic fiber formulation, the ratio may depend on the level of antimicrobial agent needed to protect the wood coupled with the amount of lubricating agent necessary for processing. It may also depend on the temperatures required and the duration that the antimicrobial composition will be exposed to those temperatures.

Thus, depending on such factors, the ratio of antimicrobial agent to amide based stabilizing agent can be very broad. As a practical matter, the 3-iodo-2-propynyl butyl carbamate and the ethylene bisamide are present in a proportion of about 1 part 3-iodo-2-propynyl butyl carbamate to 25 parts ethylene bisamide to about 1 part ethylene bisamide to 25 parts 3-iodo-2-propynyl butyl carbamate. Preferably the 3-iodo-2-propynyl butyl carbamate and the ethylene bisamide are present in a proportion of about 1 part 3-iodo-2-propynyl butyl carbamate to 10 parts ethylene bisamide to about 1 part ethylene bisamide to 10 parts 3-iodo-2-propynyl butyl carbamate. In those instances wherein minimizing the percentage of antimicrobial agent subject to degradation is of paramount importance, it is more preferable that the 3-iodo-2-propynyl butyl carbamate and the ethylene bisamide are present in a proportion of about 1 part 3-iodo-2-propynyl butyl carbamate to 15 parts ethylene bisamide to about 1 part of 3-iodo-2-propynyl butyl carbamate to 7 parts ethylene bisamide

Additives may also be optionally included in the antimicrobial composition providing that the additive does not adversely affect the antimicrobial activity of the antimicrobial agent. Non-limiting illustrative examples of additives include coupling agents, compatabilizing agents, mixing agents, viscosity stabilizers, inorganic fillers, processing aids, and coloring agents. These additives may be present in an amount from about 0.01% to about 20%, based on the total weight of the composition to achieve improvements in the physical, mechanical and thermal characteristics of the composition. A preferred compatabilizer additive is maleated polypropylene.

In a specific embodiment, the present invention provides a method of thermally stabilizing an antimicrobial agent. The method comprises admixing a antimicrobial agent with a thermally stabilizing effective amount of an amide based stabilizing agent. The thermal stability of the antimicrobial agent is improved by the presence of the stabilizing agent.

The thermally stabilized antimicrobial compositions of the present invention will generally be formulated by admixing the antimicrobial agent with the stabilizing agent. The antimicrobial agent and the stabilizing agent may be admixed as solids or the stabilizing agent may preferably be melted before it is admixed with the antimicrobial agent. Premelting the stabilizing agent before admixing it with the antimicrobial agent will result in a more homogeneous antimicrobial composition. Even if the stabilizing agent is not premelted, however, subsequent heating of the antimicrobial composition, such as in an extrusion process, will usually melt the stabilizing agent to form a more homogeneous mixture.

In another specific embodiment, the present invention provides a polymeric-cellulosic fiber formulation comprising a polymeric-cellulosic fiber composition and an antimicrobially effective amount of an antimicrobial composition. The antimicrobial composition comprises an antimicrobial agent and a thermally stabilizing effective amount of the amide based stabilizing agent to thermally stabilize the antimicrobial agent and thereby impart antimicrobial activity to the polymeric-cellulosic fiber formulation.

In this embodiment, the antimicrobial composition, comprising the antimicrobial agent and a thermally stabilizing effective amount of the stabilizing agent, is admixed with a polymeric-cellulosic fiber composition to impart antimicrobial protection to the resulting polymeric-cellulosic fiber formulation. Polymeric-cellulosic fiber compositions are wood substitutes, which generally comprise from about 30% to about 70% polyolefin or polyvinyl polymer admixed with from about 70% to about 30% cellulosic fiber. Cellulosic fibers include wood and wood products, such as wood pulp fibers, non-woody paper-making fibers from cotton, from straws and grasses, such as rice and esparto, from canes and reeds, such as bagasse, from bamboos, from stalks with bast fibers, such as jute, flax, kenaf, cannabis, linen and ramie, and from leaf fibers, such as abaca and sisal. The wood flours used in the polymeric-cellulosic fiber compositions include soft and hard woods such as oak, pine, and maple.

Polymeric-cellulosic fiber formulations are generally prepared by extrusion methods. The polymeric-cellulosic fiber composition and the antimicrobial composition are mixed in a mixer and then dried at elevated temperatures under vacuum. The dried compositions are then extruded at temperatures typically about 150° C. The extruded material is typically passed through a cooling chamber containing water sprays before being cut and collected. Addition of the thermally stabilized antimicrobial composition to the polymeric-cellulosic fiber composition will provide antimicrobial properties in the final extruded formulation even after high temperature extrusion.

In another specific embodiment, the present invention provides an improved method for making a polymeric-cellulosic fiber formulation with antimicrobial protection. The method comprises adding an antimicrobially effective amount of an antimicrobial composition to a polymeric-cellulosic fiber composition. The antimicrobial composition comprises an admixture of the antimicrobial agent and a thermally stabilizing effective amount of the stabilizing agent, to thermally stabilize the antimicrobial agent and thereby impart antimicrobial protection to the polymeric-cellulosic fiber formulation.

In this embodiment, the improved method for making a polymeric-cellulosic fiber formulation with antimicrobial protection comprises adding an antimicrobially effective amount of a antimicrobial composition to a polymeric-cellulosic fiber composition. The antimicrobial composition comprises an antimicrobial agent and a thermally stabilizing effective amount of a stabilizing agent, to thermally stabilize the antimicrobial agent and thereby impart antimicrobial protection to the polymeric-cellulosic fiber formulation.

In addition to extrusion, the polymeric-cellulosic fiber formulations of this invention may be injection molded to produce commercially usable products. The resultant product has an appearance similar to wood and may be sawed, sanded, shaped, or finished in the same manner as natural wood. The products are resistant to rot and decay and may be used as interior or exterior decorative moldings, furniture, porch decks, window moldings, window components, door components, and other structural members.

A particularly preferred aspect of the present invention relates to an antimicrobial composition containing an antimicrobial agent and a thermally stabilizing effective amount of an amide based stabilizing agent which can be sold as a concentrate and which is useful as an antimicrobial additive for introducing the antimicrobial agent into an end-use formulation.

The following examples are presented to illustrate and explain the invention. Unless otherwise indicated, all references to parts and percentages here and throughout the application are based on weight.

### EXAMPLES

### Examples 1-5

These Examples illustrate the effect of various lubricating agents on the thermal stability of the antimicrobial agent, 3-iodo-2-propynyl butyl carbamate. The data in Table 1 show that the amide based stabilizing agent which is also a lubricating agent, Glycolube® WP2200, provides thermal stability to the antimicrobial agent IPBC.

Except for example 2 wherein 100% IPBC was used, the antimicrobial agent was admixed with the lubricating agent at room temperature overnight in a roller mixer. Samples of the resulting mixtures were then placed in a conventional oven at 180° C. for the time specified below (5 minutes and 132 minutes). The amount of antimicrobial agent in the sample, initially and after heating, was determined by HPLC. All samples were contained in dark bottles and covered with aluminum foil during heating.

TABLE 1

| Example | 1 | 2 | 3 | 4 | 5 |
|---|---|---|---|---|---|
| Biocide | IPBC | IPBC | IPBC | IPBC | IPBC |
| Lubricant | G | | G | TX | Zn |
| % Initial Biocide | 13 | 100 | 15.2 | 0.19 | 12 |
| % Biocide/ 5 min. | 13 | 83 | 12.7 | 0.18 | 12 |
| % Biocide/ 132 min. | 6.9 | 0 | 5.9 | 0 | 0 |
| % Recovery/ 5 min. | 100 | 83 | 84 | 95 | 100 |
| % Recovery/ 132 min. | 53 | 0 | 39 | 0 | 0 |

IPBC is 99.7% 3-iodo-2-propynyl butyl carbamate.
G = Glycolube ® WP2200, Lonza, mp. 146° C.
TX = complex modified fatty acid esters (C8-C18) (Struktol, mp. 72°C.).
Zn = Zinc stearate (Ferro Corporation (#8), mp. 120° C.).

Table 1 shows that under the most harsh conditions that the IPBC was totally destroyed in all cases where there was no amide based stabilizing agent present. Only those samples having the amide based stabilizing agent Glycolube® WP2200 were thermally stable at 180° C. for 132 minutes.

### Examples 6-11

These Examples illustrate the effect of the concentration of the, Glycolube® WP2200, on the thermal stability of the antimicrobial agent, 3-iodo-2-propynyl butyl carbamate. The data in Table 2 suggest that the most efficient thermal protection to the antimicrobial agent is in the concentration ranges near 13%.

The samples were prepared and tested as described in Examples 1-5.

TABLE 2

| Example | 6 | 7 | 8 | 9 | 10 | 11 |
|---|---|---|---|---|---|---|
| Biocide | IPBC | IPBC | IPBC | IPBC | IPBC | IPBC |
| Lubricant | G | G | G | G | G | G |
| % Initial Biocide | 46 | 33 | 20 | 14 | 13 | 10 |
| % Biocide/ 5 min. | 42 | 32 | 20 | 13 | 13 | 10 |
| % Biocide/ 132 min. | 14.85 | 8.7 | 8.8 | 7.5 | 6.9 | 4.9 |
| % Recovery/ 5 min. | 91 | 97 | 100 | 93 | 100 | 100 |
| % Recovery/ 132 min. | 32.3 | 26.4 | 44 | 54 | 53 | 49 |

IPBC is 99.7% 3-iodo-2-propynyl butyl carbamate.
G = Glycolube ® WP2200, Lonza, mp. 146° C.

### Examples 12-14

These Examples illustrate the effect on the antimicrobial activity of heating the antimicrobial agent, 3-iodo-2-propynyl butyl carbamate, in the presence of the lubricating agent, Glycolube® WP2200. The data in Table 3 shows that the level of antimicrobial activity is consistent with the analytical analysis and that the antimicrobial activity was retained even after 132 minutes at 180° C.

The samples were prepared and heated as described in Examples 1-5. The samples were then diluted with THF to form a 0.5% solution. Half-inch filter paper discs were then dipped into the samples. The discs were then dried for 5 hours and then placed on 2.5% malt agar plates previously seeded with conidia from Aspergillus niger. The plates were then incubated for 24 hours at 27° C. and the zone of inhibition was measured with a caliper.

TABLE 3

| | | | Inhibition |
|---|---|---|---|
| Example | % Initial Biocide | Treatment Conditions | Zone(mm) |
| 12 | 15.2 | Initial | 28 |
| 13 | 12.7 | 180° C. for 5 min. | 27 |
| 14 | 5.9 | 180° C. for 132 min. | 10 |

While the invention has been illustrated by reference to specific and preferred embodiments, those skilled in the art will understand that variations and modifications may be made through routine experimentation and practice of the invention. Thus, the invention is intended not to be limited by the foregoing description, but to be defined by the appended claims and their equivalents.

We claim:

1. An antimicrobial composition comprising 3-iodo-2-propynyl butyl carbamate and an effective amount amide based stabilizing agent.

2. The antimicrobial composition of claim 1 wherein the amide based stabilizing agent is an ethylene bisamide.

3. The composition of claim 2, wherein the ethylene bisamide is represented by the formula:
R—CO—N(H)—CH₂CH₂—N(H)—CO—R
wherein R is independently an alkyl group having from 6 to 16 carbon atoms.

4. The composition of claim 3 wherein 3-iodo-2-propynyl butyl carbamate and the ethylene bisamide are presented in a proportion of about 1 part 3-iodo-2-propynyl butyl carbamate to 25 parts ethylene bisamide to about 1 part ethylene bisamide to 25 parts 3-iodo-2-propynyl butyl carbamate.

5. The composition of claim 4, wherein R is independently an alkyl group having from 7 to 13 carbon atoms.

6. The composition of claim 5, wherein the ethylene bisamide agent is selected from the group consisting of ethylene bisoctanamide, ethylene bisdecanamide, ethylene bisdodecanamide, ethylene bistetradecanamide and combinations thereof.

7. The composition of claim 6 wherein 3-iodo-2-propynyl butyl carbamate and the ethylene bisamide are presented in a proportion of about 1 part 3-iodo-2-propynyl butyl carbamate to 10 parts ethylene bisamide to about 1 part ethylene bisamide to 10 parts 3-iodo-2-propynyl butyl carbamate.

8. A polymeric-cellulosic fiber formulation in which there has been incorporated a thermally stabilized antimicrobial composition comprising an antimicrobially effective amount of 3-iodo-2-propynyl butyl carbamate and a thermally stabilizing effective amount of an ethylene bisamide.

9. The formulation of claim 8, wherein the ethylene bisamide is represented by the formula:
R—CO—N(H)—CH₂CH₂—N(H)—CO—R
wherein R is independently an alkyl group having from 6 to 16 carbon atoms.

10. The composition of claim 9 wherein 3-iodo-2-propynyl butyl carbamate and the ethylene bisamide are presented in a proportion of about 1 part 3-iodo-2-propynyl butyl carbamate to 25 parts ethylene bisamide to about 1 part ethylene bisamide to 25 parts 3-iodo-2-propynyl butyl carbamate.

11. The composition of claim 10 wherein R is independently an alkyl group having from 7 to 13 carbon atoms.

12. The composition of claim 11, wherein the ethylene bisamide agent is selected from the group consisting of ethylene bisoctanamide, ethylene bisdecanamide, ethylene bisdodecanamide, ethylene bistetradecanamide and combinations thereof.

13. The composition of claim 12 wherein 3-iodo-2-propynyl butyl carbamate and the ethylene bisamide are presented in a proportion of about 1 part 3-iodo-2-propynyl butyl carbamate to 10 parts ethylene bisamide to about 1 part ethylene bisamide to 10 parts 3-iodo-2-propynyl butyl carbamate.

14. A method of thermally stabilizing 3-iodo-2-propynyl butyl carbamate, which comprises admixing the 3-iodo-2-propynyl butyl carbamate with an ethylene bisamide.

15. The method of claim 14, wherein the ethylene bisamide is represented by the formula:
R—CO—N(H)—CH₂CH₂—N(H)—CO—R
wherein R is independently an alkyl group having from 6 to 16 carbon atoms.

16. The method of claim 15, wherein the ethylene bisamide is melted prior to being admixed with the 3-iodo-2-propynyl butyl carbamate.

17. An improved method for making a polymeric-cellulosic fiber formulation with antimicrobial protection which comprises adding an antimicrobially effective amount of a 3-iodo-2-propynyl butyl carbamate to a polymeric-cellulosic fiber composition, wherein the antimicrobial composition comprises an admixture of a 3-iodo-2-propynyl butyl carbamate and a thermally stabilizing effective amount of an ethylene bisamide, to thermally stabilize the 3-iodo-2-propynyl butyl carbamate and thereby impart antimicrobial protection to the polymeric-cellulosic fiber formulation.

18. The method of claim 17, wherein the ethylene bisamide is represented by the formula:
R—CO—N(H)—CH₂CH₂—N(H)—CO—R
wherein R is independently an alkyl group having from 6 to 16 carbon atoms.

19. The method of claim 17, wherein the ethylene bisamide is melted prior to being admixed with the 3-iodo-2-propynyl butyl carbamate.
